(19) United States
(12) Patent Application Publication
(10) Pub. No.: US 2023/0072604 A1
(43) Pub. Date: Mar. 9, 2023

(54) SYSTEM AND METHOD FOR MITIGATING SIDE EFFECTS OF CHEMOTHERAPY

(71) Applicant: EISANA CORPORATION, The Woodlands, TX (US)

(72) Inventors: Carole SPANGLER VAUGHN, Spring, TX (US); Brian Patrick WILFLEY, Sunnyvale, CA (US); William WELCH, Sunnyvale, CA (US); Hamilton Roger TANG, Los Altos, CA (US); Todd L. HARRIS, Fremont, CA (US)

(73) Assignee: EISANA CORPORATION, The Woodlands, TX (US)

(21) Appl. No.: 17/759,593

(22) PCT Filed: Jan. 28, 2021
(86) PCT No.: PCT/US2021/015538

§ 371 Date: Jul. 27, 2022

Related U.S. Application Data
(60) Provisional application No. 62/966,912, filed on Jan. 28, 2020.

(51) Int. Cl. A61F 7/02

(52) U.S. Cl. CPC A61F 7/02; A61F 2007/0002

(57) ABSTRACT

A therapeutic cooling and/or compression system, configured to cool a body portion, comprises a conformal covering for covering the body portion configured to extract heat energy from the body portion, a sensor device within the conformal covering for sensing a parameter of the body portion, an actuator configured for changing an amount of heat energy extracted from the body portion, and a control unit configured for regulating the actuator responsive to control input from the sensor device. The system may be portable, enabling a patient to walk while undergoing treatment.

### RELATED APPLICATION(S)

This application claims priority to U.S. Provisional Application 62/966,912, “Method and Device for Scalp Cooling,” to Spangler Vaughn, filed 28 Jan. 2020, which is hereby incorporated herein by reference in its entirety.

### FIELD OF INVENTION

Embodiments of the present invention relate to the field of medical devices. More specifically, embodiments of the present invention relate to systems and methods for mitigating side effects of chemotherapy.

### BACKGROUND

Chemotherapy describes or refers to a drug treatment that uses powerful chemicals to kill fast-growing cells. Chemotherapy is often used to treat a variety of cancers, as cancer cells generally grow and multiply much more quickly than most cells in the body. For medical patients undergoing chemotherapy, the toxicity of the chemotherapy drug(s) may cause a number of problems (side effects) that make the treatment hard to tolerate. Some common side effects are hair loss, including from the head, peripheral neuropathy, oral mucositis (inflammation and ulceration of the mucous membranes), and oncolysis (detachment of the nail from the nail bed).

Several studies report that loss of scalp hair is one of the most distressing side effects. One study reported that up to 8% of women will refuse chemotherapy because of hair loss (Tierney A J, Taylor J, Closs S J, Knowledge, expectations and experiences of patients receiving chemotherapy for breast cancer, Scand J Caring Sci 1992:6:75-80). Some patients may forego chemotherapy due to the perceived severity of such side effects. Some patients may require and/or demand a change and/or reduction of chemotherapy in view of anticipated or experienced side effects, detrimentally reducing the therapy's effectiveness.

### SUMMARY OF THE INVENTION

Therefore, what is needed are systems and methods for mitigating side effects of chemotherapy. What is additionally needed are systems and methods for mitigating side effects of chemotherapy that are portable and allow a patient to walk and/or be transported, for example, in a car or bus during treatment. Further, there is a need for systems and methods for mitigating side effects of chemotherapy that provide a patient and/or caregiver with temperature and/or pressure control. There is a still further need for systems and methods for mitigating side effects of chemotherapy that are compatible and complementary with existing systems and methods of administering chemotherapy.

In accordance with an embodiment of the present invention, a therapeutic cooling system, which may also include an ability to control compression, configured to cool and/or compress a body portion, comprises a conformal covering for covering the body portion configured to extract heat energy from the body portion, a sensor device within the conformal covering for sensing a parameter of the body portion, an actuator configured for changing an amount of heat energy extracted from the body portion, and a control unit configured for regulating the actuator responsive to control input from the sensor device. The system may be portable, enabling a patient to walk or be transported while undergoing treatment. The actuator may be configured for changing an amount of compression applied to the body portion.

### BRIEF DESCRIPTION OF THE DRAWINGS

The accompanying drawings, which are incorporated in and form a part of this specification, illustrate embodiments of the invention and, together with the description, serve to explain the principles of the invention. Unless otherwise noted, the drawings may not be drawn to scale.

FIG. 1 illustrates a block diagram of an exemplary closed loop controller for use with a heating/cooling/compression apparatus, in accordance with embodiments of the present invention.

FIG. 2 illustrates a block diagram of an exemplary gas-based heating/cooling/compression system, in accordance with embodiments of the present invention.

FIG. 3 illustrates a block diagram of an exemplary gas-based heating/cooling/compression system, in accordance with embodiments of the present invention.

FIG. 4 illustrates a block diagram of an exemplary thermoelectric cooler-based heating/cooling/compression system, in accordance with embodiments of the present invention.

FIG. 5 illustrates a block diagram of an exemplary thermoelectric cooler-based heating/cooling/compression system, in accordance with embodiments of the present invention.

FIG. 6 illustrates a block diagram of an exemplary heating/cooling/compression system, in accordance with embodiments of the present invention.

FIG. 7 illustrates a block diagram of an exemplary heating/cooling/compression system, in accordance with embodiments of the present invention.

FIG. 8 illustrates a block diagram of an exemplary multi zone thermal management system, in accordance with embodiments of the present invention.

FIG. 9 illustrates a block diagram of an exemplary multi zone thermal management system, in accordance with embodiments of the present invention.

FIG. 10 illustrates a block diagram of an exemplary heating/cooling/compression system, in accordance with embodiments of the present invention.

FIG. 11 illustrates a block diagram of an exemplary heating/cooling/compression system, in accordance with embodiments of the present invention.

FIG. 12 illustrates a block diagram of an exemplary heating/cooling/compression system, in accordance with embodiments of the present invention.

FIG. 13 illustrates a block diagram of an exemplary heating/cooling/compression system, in accordance with embodiments of the present invention.

FIG. 14 illustrates a block diagram of an exemplary heating/cooling/compression system, in accordance with embodiments of the present invention.

FIG. 15 illustrates a block diagram of an exemplary thermal safety system, in accordance with embodiments of the present invention.

FIG. 16 illustrates a block diagram of an exemplary thermal control system, in accordance with embodiments of the present invention.

FIG. 17 illustrates a block diagram of an exemplary thermal management system, in accordance with embodiments of the present invention.

FIG. 18 illustrates a block diagram of an exemplary hair management system, in accordance with embodiments of the present invention.

FIG. 19 illustrates a block diagram of an exemplary hair management system, in accordance with embodiments of the present invention.

FIG. 20 illustrates a block diagram of an exemplary hair management system, in accordance with embodiments of the present invention.

FIG. 21 illustrates a block diagram of an exemplary hair management system, in accordance with embodiments of the present invention.

FIG. 22 illustrates a block diagram of an exemplary hair management system, in accordance with embodiments of the present invention.

FIG. 23 illustrates a block diagram of an exemplary hair management system, in accordance with embodiments of the present invention.

FIG. 24 illustrates a block diagram of an exemplary closed loop controller for use with a heating/cooling/compression apparatus, in accordance with embodiments of the present invention.

FIGS. 25A, 25B, and 25C illustrate exemplary systems of heating, cooling, and/or compressing hands, fingers and arms, respectively, in accordance with embodiments of the present invention.

FIGS. 26A and 26B illustrate exemplary systems of heating, cooling, and/or compressing legs and/or feet, in accordance with embodiments of the present invention.

FIG. 27 illustrates an exemplary heating/cooling/compression apparatus liner, in accordance with embodiments of the present invention.

FIGS. 28A and 28B illustrate exemplary liners, in accordance with embodiments of the present invention.

FIG. 29 illustrates an exemplary heating/cooling/compression apparatus, in accordance with embodiments of the present invention.

FIG. 30 illustrates an exemplary heating/cooling/compression apparatus, in accordance with embodiments of the present invention.

FIG. 31 illustrates a block diagram of an exemplary electronic system, which may be used as a platform to implement and/or as a control system for embodiments of the present invention.

FIG. 32 illustrates an exemplary method for mitigating side effects of chemotherapy, in accordance with embodiments of the present invention.

### DETAILED DESCRIPTION

Reference will now be made in detail to various embodiments of the invention, examples of which are illustrated in the accompanying drawings. While the invention will be described in conjunction with these embodiments, it is understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the invention, numerous specific details are set forth in order to provide a thorough understanding of the invention. However, it will be recognized by one of ordinary skill in the art that the invention may be practiced without these specific details. In other instances, well known methods, procedures, components, and circuits have not been described in detail as not to unnecessarily obscure aspects of the invention.

Some portions of the detailed descriptions which follow (e.g., method 3200) are presented in terms of procedures, steps, logic blocks, processing, and other symbolic representations of operations on data bits that may be performed on computer memory. These descriptions and representations are the means used by those skilled in the data processing arts to most effectively convey the substance of their work to others skilled in the art. A procedure, computer executed step, logic block, process, etc., is here, and generally, conceived to be a self-consistent sequence of steps or instructions leading to a desired result. The steps are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, data, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present invention, discussions utilizing terms such as “testing” or “heating” or “maintaining temperature” or “bringing” or “capturing” or “storing” or “reading” or “analyzing” or “generating” or “resolving” or “accepting” or “selecting” or “determining” or “displaying” or “presenting” or “computing” or “sending” or “receiving” or “reducing” or “detecting” or “setting” or “accessing” or “placing” or “testing” or “forming” or “mounting” or “removing” or “ceasing” or “stopping” or “coating” or “processing” or “performing” or “generating” or “adjusting” or “creating” or “executing” or “continuing” or “indexing” or “translating” or “calculating” or “measuring” or “gathering” or “running” or the like, refer to the action and processes of, or under the control of, a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The meaning of “non-transitory computer-readable medium” should be construed to exclude only those types of transitory computer-readable media which were found to fall outside the scope of patentable subject matter under 35 U.S.C. § 101 in In re Nuijten, 500 F.3d 1346, 1356-57 (Fed. Cir. 2007). The use of this term is to be understood to remove only propagating transitory signals per se from the claim scope and does not relinquish rights to all standard computer-readable media that are not only propagating transitory signals per se.

In the following descriptions, various elements and/or features of embodiments in accordance with the present invention are presented in isolation so as to better illustrate such features and as not to unnecessarily obscure aspects of the invention. It is to be appreciated, however, that such features, e.g., as disclosed with respect to a first drawing, may be combined with other features disclosed in other drawings in a variety of combinations. All such embodiments are anticipated and considered, and may represent embodiments in accordance with the present invention.

### System and Method for Mitigating Side Effects of Chemotherapy

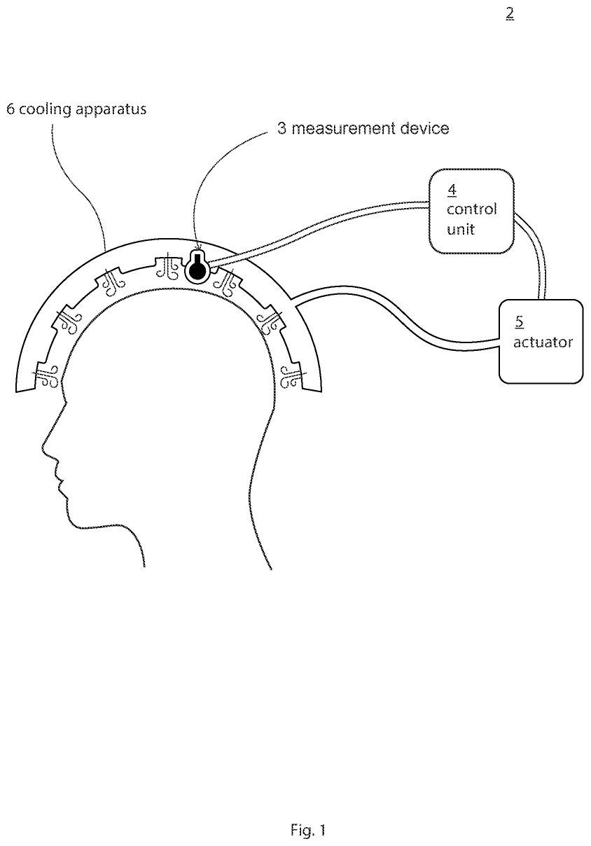

FIG. 1 illustrates a block diagram of an exemplary closed loop controller 2 for use with a heating/cooling/compression apparatus, e.g., a cooling cap 6, in accordance with embodiments of the present invention. The heating/cooling/compression apparatus is configured to conform to a particular body portion, e.g., a human head. As will be further described below, embodiments in accordance with the present invention are not limited to cooling, heating, and/or compressing the head. Rather, embodiments in accordance with the present invention are applicable to application to any appropriate body part(s), including, for example, arms, legs, torso, hips, hands, fingers, fingertips, ears, feet, toes, face, lips, eyebrows, and/or the tongue.

Controller 2 comprises a measurement device 3, a control unit 4, and an actuator 5. The measurement device 3 may comprise a thermistor, a fiber-optic thermometer, a non-contact thermometer, e.g., an infrared thermometer, or other temperature measurement device(s). The measurement device 3 may measure the temperature of the skin, the temperature of the medium, such as air or a fluid or gel proximate to the skin, the temperature of hair, the temperature of a covering or garment proximate to the skin, or any combination of these.

The control unit 4 may be a thermostat, an electronic controller such as a PID (proportional-integral-derivative) controller, a computerized control unit, or any other control device. The actuator 5 may be any apparatus for controlling the degree of heating/cooling/compression applied to the skin or the space immediately proximate to the skin. The controller may be programmed by receiving input from the caregiver or the patient as to the drug being administered and additional information about the treatment protocol or patient demographic information. Such programming may allow the heating/cooling/compression protocol to be adapted to the patient, drug, or treatment protocol so as to optimize the outcome of heating, cooling, and/or compression. The control unit 4 may provide for changing, for example, ramping, the temperature and/or compression of the skin at a finite and/or customized rate to prevent shock to the skin. Such control may be provided independently for either cooling or warming or compression of the skin. The controller may provide safety systems and methods of ensuring safety of tissues of and beneath the skin by altering an amount of cooling, compression, and/or heating.

In accordance with embodiments of the present invention, measurement device 3 may sense properties of tissue being cooled, e.g., rather than or in addition to directly sensing temperature. Such a sensor may sense blood flow, pulse and/or pulse rate, a presence or absence of substances, cellular metabolism, e.g., via NADH (nicotinamide adenine dinucleotide and hydrogen) fluorescence sensors, pressure and/or compression, or any other suitable sensed property. Sensed substances may include chemotherapy agents and/or their metabolites, or other substances indicative of tissue status or tissue metabolism. The output from any such sensor may be incorporated into control such that changes in a sensed property cause corresponding changes in the control of cooling, heating, and/or compression. Such incorporation of the output of any such sensor into control may allow control to alter cooling, heating, and/or compression to avoid unsafe conditions for such tissue being cooled. In some embodiments sensors may sense properties of the head or the tissues of limbs such as arms, legs, hands, feet, fingers, face, eyebrows, toes, lips and/or mouth, or any combination thereof.

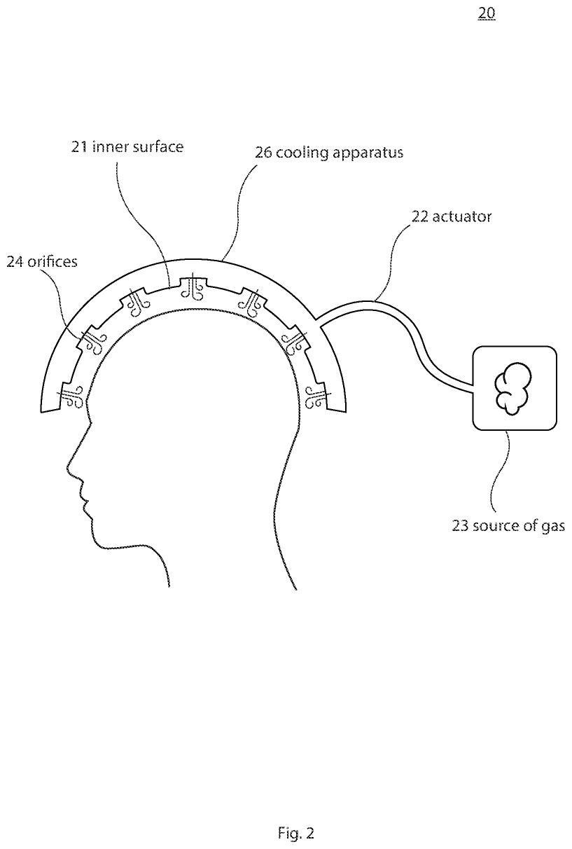

FIG. 2 illustrates a block diagram of an exemplary gas-based heating/cooling/compression system 20, in accordance with embodiments of the present invention. Heating/cooling/compression system 20 comprises a heating/cooling/compression apparatus 26, e.g., a cooling cap, for application over a head or scalp. The heating/cooling/compression apparatus 26 is configured to conform to a particular body portion, e.g., a human head. The apparatus 26 may be characterized as having an inner surface 21, proximate to the skin to be cooled. The inner surface 21 may be penetrated by an array (regular or irregular) of small orifices 24, which are designed to allow a compressed gas to be emitted into the narrow space between the inner surface and the scalp. The gas, such as nitrogen, argon, carbon dioxide, or other gas may be supplied from a source of gas 23, such as a gas bottle (pressure tank) or chemical gas generator. The source of gas may be coupled to the orifices, e.g., by a tube or pipe, and/or by either branching tubes and/or by a plenum.

The pressurized gas, upon being emitted by the orifices, cools due to the Joule-Thompson effect. The emitted cooled gas passes close to the skin as it escapes the narrow space, thus heating, cooling, and/or compressing the skin. In some embodiments, temperature control may utilize heating and/or cooling of the gas at its source 23. Control of the temperature may be achieved by varying the rate of flow of the gas. Such variation can be achieved by an actuator 22, such as a valve, a variable pump, or by adjusting the rate at which a chemical gas generator generates gas. In some embodiments, the source of gas 23 may be portable allowing the patient to move from place to place. It is appreciated that the embodiments of FIG. 2 are applicable to temperature control in accordance with the embodiments of FIG. 1.

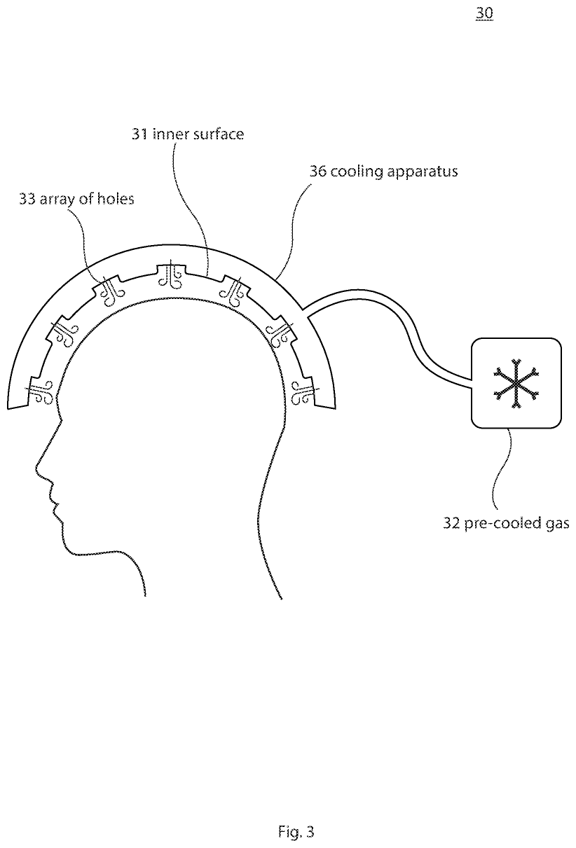

FIG. 3 illustrates a block diagram of an exemplary gas-based heating/cooling/compression system 30, in accordance with embodiments of the present invention. Heating/cooling/compression system 30 comprises a heating/cooling/compression apparatus 36, e.g., a cooling cap. The heating/cooling/compression apparatus 36 is configured to conform to a particular body portion, e.g., a human head. Heating/cooling/compression apparatus 36 comprises an inner surface 31 and an array of holes 33. A pre-cooled gas 32 may be coupled to the array of holes 33 such that the gas 32 may flow onto the skin.

The inner surface may be penetrated by a plurality or array (regular or irregular) of holes 33, which are designed to allow a gas to be emitted into the narrow space between the inner surface and the skin. Some embodiments may further comprises a gas supply 32 that supplies pre-cooled gas to the holes in the cap. The pre-cooled gas supply 32 may be connected to the holes 33 in the cap by a tube, a plenum, or other connection. The emitted pre-cooled (or heated) gas passes close to the skin as it escapes the narrow space, thus cooling (heating) the skin. Control of the temperature may be achieved by varying the rate of flow of the gas 32 or the temperature of the pre-cooled gas 32. Such variation may be achieved by an actuator, such as a valve, a variable pump, or other control system. In some embodiments, a pre-cooled gas may be additionally cooled by the Joule-Thompson effect. In some embodiments, the pre-cooled gas supply 32 may be portable allowing the patient to move from place to place. It is appreciated that the embodiments of FIG. 3 are applicable to temperature control in accordance with the embodiments of FIG. 1.

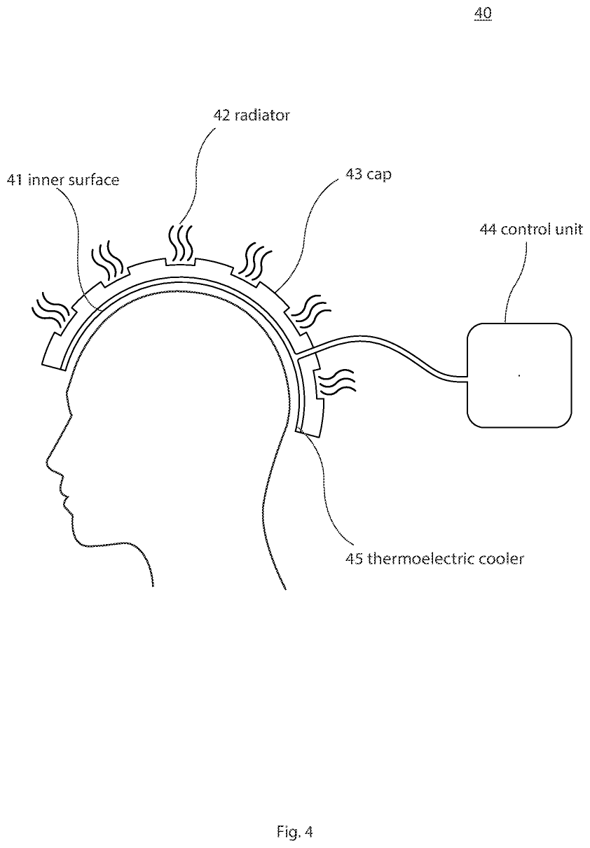

FIG. 4 illustrates a block diagram of an exemplary thermoelectric cooler (TEC)-based heating/cooling/compression system 40, in accordance with embodiments of the present invention. Heating/cooling/compression system 40 comprises a heating/cooling/compression apparatus 43, e.g., a cooling cap, and a control unit 44. The heating/cooling/compression apparatus 43 is configured to conform to a particular body portion, e.g., a human head. The heating/cooling/compression apparatus 43 comprises an inner surface 41, a convective component 42, e.g., a radiator, and a plurality of thermoelectric coolers 45. The thermoelectric coolers may comprise Peltier devices, in some embodiments.

The heating/cooling/compression apparatus 43 fits onto the body, e.g., head, hands, fingers, feet, and/or toes, with an inner surface 41 proximate to the skin. The heating/cooling/compression apparatus 43 comprises a plurality of thermoelectric coolers 45 that removes heat from the surface proximate to the skin, thus cooling the skin. In some embodiments, the thermoelectric coolers 45 may apply heat to the surface proximate to the skin, thus heating the skin. The cap may further comprise a radiative or convective component 42 that receives the removed heat from the plurality of thermoelectric coolers 45 and dumps that heat into the environment around the patient such as the room air. In some embodiments, the control unit 44 may be portable allowing the patient to move from place to place. It is appreciated that the embodiments of FIG. 4 are applicable to temperature control in accordance with the embodiments of FIG. 1.

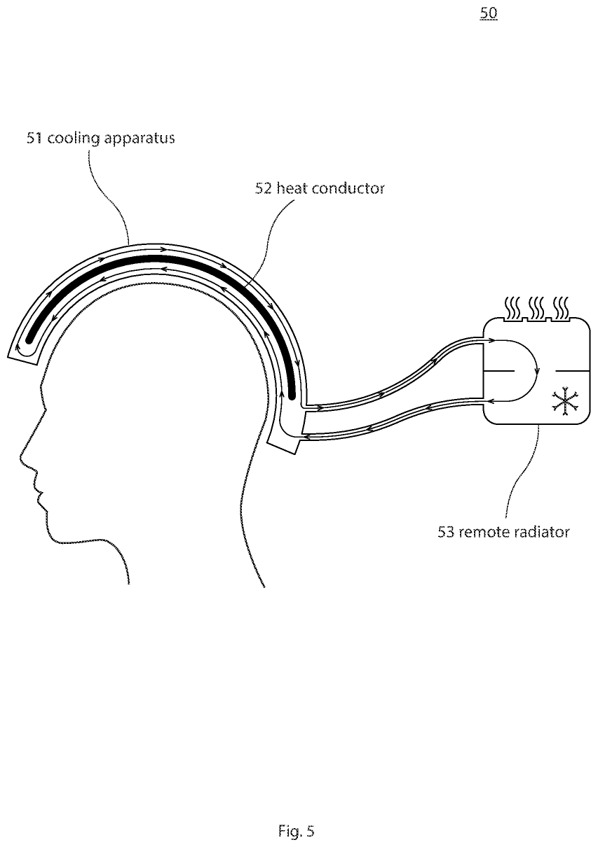

FIG. 5 illustrates a block diagram of an exemplary thermoelectric cooler (TEC)-based heating/cooling/compression system 50, in accordance with embodiments of the present invention. Heating/cooling/compression system 50 comprises a heating/cooling/compression apparatus 51, e.g., a cooling cap, and a remote radiator 53. The heating/cooling/compression apparatus 51 is configured to conform to a particular body portion, e.g., a human head. In contrast to embodiments in accordance with FIG. 4, a radiative element 53 is not a part of the cooling apparatus 51, but rather is remote to the cooling apparatus 51. In this embodiment, a heat conductor 52 may be provided that conducts heat from the cooling apparatus 51 to the remote radiator 53. Such a conductor may comprise a metallic medium providing high thermal conductivity, for example, copper, silver, mercury, or other metals, a heat-pipe, a pulsating or loop-type heat pipe, a pumped liquid such as water, alcohol, a solution with poly-ethylene glycol, or other liquids, a pumped gas such as nitrogen, argon, carbon-dioxide, or other gases, or other systems and methods by which to conduct heat. The removed heat, once conducted to the radiator, may be dumped by the radiator into the environment. In some embodiments, the remote radiator 53 may be portable allowing the patient to move from place to place. It is appreciated that the embodiments of FIG. 5 are applicable to temperature control in accordance with the embodiments of FIG. 1.

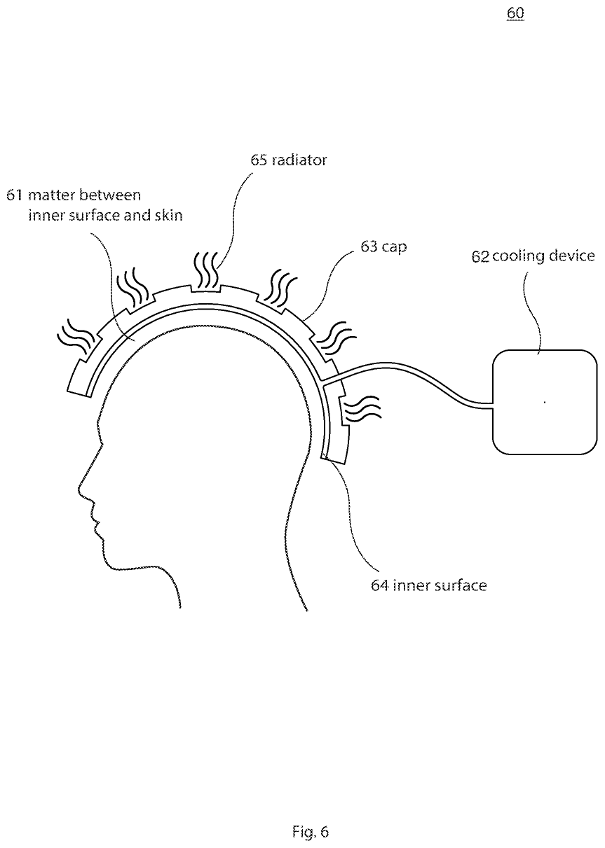

FIG. 6 illustrates a block diagram of an exemplary heating/cooling/compression system 60, in accordance with embodiments of the present invention. Heating/cooling/compression system 60 comprises a heating/cooling/compression apparatus 63, e.g., a cooling cap. The heating/cooling/compression apparatus 63 is configured to conform to a particular body portion, e.g., a human head. A thermally conductive material 61 may be placed between an inner surface 61 of the cooling apparatus 63 and the skin. The inner surface 61 may be cooled by a cooled gas, cooled liquid, a thermoelectric cooler, a phase-change cooler, or any other cooling device 62. The cap 63 may further comprise a radiative or convective component 65 that receives the removed heat from the skin area and dumps that heat into the environment around the patient such as the room air. Radiative elements 65 may comprise vents in some embodiments.

The inner surface 64 provides a cold boundary condition to the narrow space between the inner surface 64 and the skin. The matter 61 lying between the inner surface and the skin transmits heat from the skin to the inner surface, thus cooling the skin. The transmission of heat may be due to inherent properties of the matter 61 such as thermal conductance, or the tendency of the matter to form convective flow, or other properties. The matter can be reusable or disposable and may cause pressure and/or compression to the skin. It is appreciated that the embodiments of FIG. 6 are applicable to temperature control in accordance with the embodiments of FIG. 1.

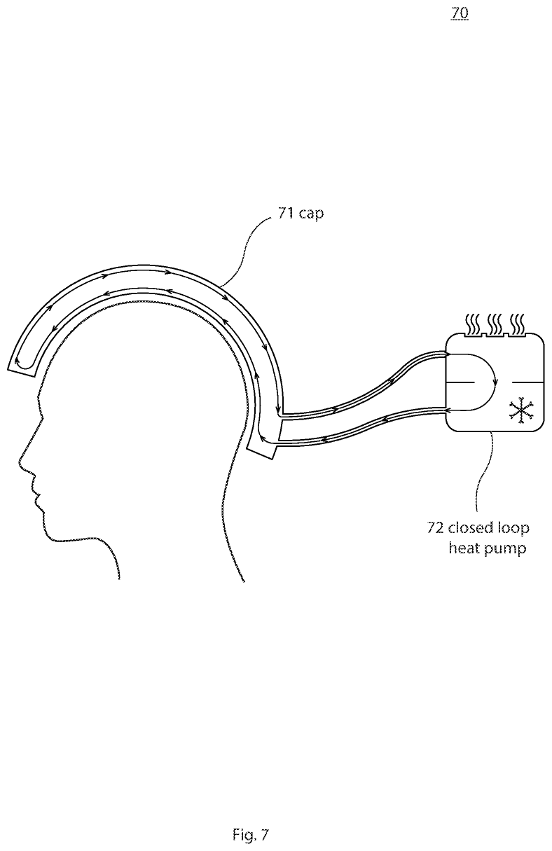

FIG. 7 illustrates a block diagram of an exemplary heating/cooling/compression system 70, in accordance with embodiments of the present invention. Heating/cooling/compression system 70 comprises a heating/cooling/compression apparatus 71, e.g., a cooling cap. The heating/cooling/compression apparatus 71 is configured to conform to a particular body portion, e.g., a human head. Cooling may be supplied, either to gas, to liquid, to the heating/cooling/compression apparatus 71, to matter between the heating/cooling/compression apparatus 71 and to the skin, e.g., matter 61 of FIG. 6, or to the skin, by a refrigerator such as a closed loop heat pump 72, for example, a Sterling-cycle refrigerator, a thermoacoustic refrigerator, a thermoelectric cooler, or other method of forcing the transport of heat. In some embodiments, the closed loop heat pump 72 may be portable allowing the patient to move from place to place. It is appreciated that the embodiments of FIG. 7 are applicable to temperature control in accordance with the embodiments of FIG. 1.

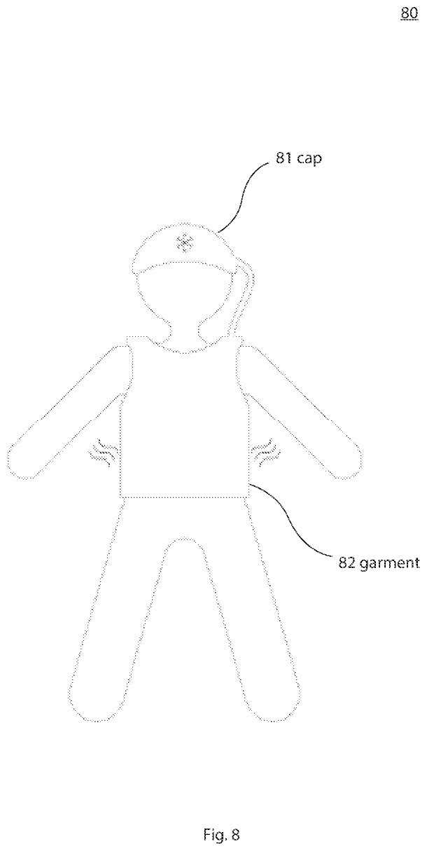

FIG. 8 illustrates a block diagram of an exemplary multi zone thermal management system 80, in accordance with embodiments of the present invention. System 80 comprises a heating/cooling/compression apparatus 71, e.g., a cooling cap and a warming apparatus 82, e.g., a garment. The heating/cooling/compression apparatuses 71, 82 are configured to conform to a particular body portion, e.g., a human head and/or torso. In accordance with embodiment of the present invention, heat removed due to the cooling of one or more body parts, e.g., the scalp, may be supplied to other regions of the patient's body for the purpose of keeping the patient warm. For example, a fluid may transport heat extracted from a scalp to other body areas. The heat may be supplied by warm air blowing on the patient and/or by a liquid, to a warming apparatus 82, such as a vest, leg-warmers, socks, booties, slippers, gloves or mittens, sleeves, cuffs, and/or a mask, that are proximate to the skin of the body, or by other systems of keeping the patient warm. It is appreciated that the embodiments of FIG. 8 are applicable to temperature control for both heating and cooling in accordance with the embodiments of FIG. 1.

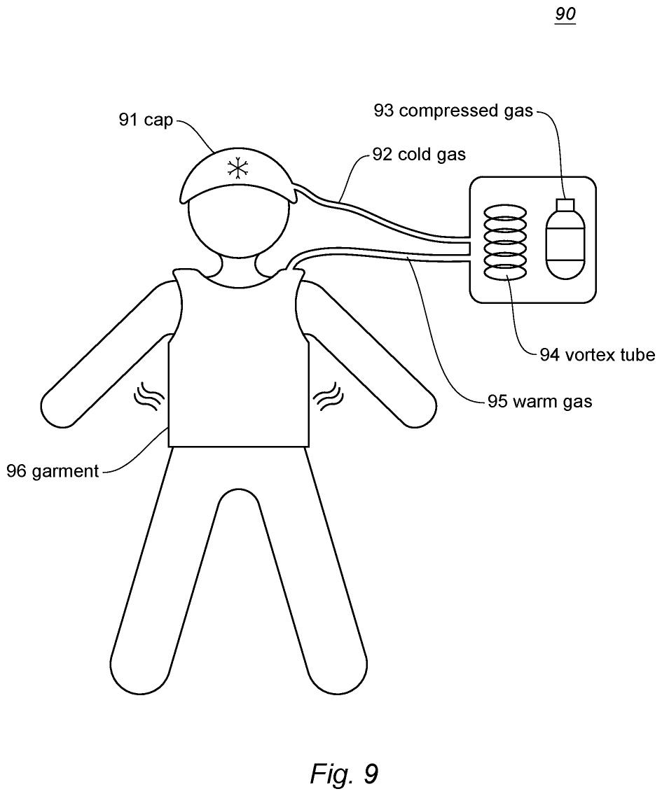

FIG. 9 illustrates a block diagram of an exemplary multi zone thermal management system 90, in accordance with embodiments of the present invention. System 90 comprises a heating/cooling/compression apparatus 91, e.g., a cooling cap, a cold gas 92, a container of compressed gas 93, a vortex tube 94, a warm gas 95, and a warming apparatus 96, e.g., a garment. The heating/cooling/compression apparatuses 91, 96 are configured to conform to a particular body portion, e.g., a human head and/or torso. The cooling apparatus 91 and/or the warming apparatus 96 may comprise a cap or hat, a vest, leg warmers, socks, booties, slippers, gloves or mittens, sleeves, cuffs, and/or a mask, in some embodiments.

In accordance with embodiments of the present invention, one or more portions of a body, e.g., the head, may be cooled, while other portion(s) of the body, e.g., the torso, are warmed. Cold gas 92 may be supplied to cool a portion of the body, and warm gas 95 may be supplied a warm a portion of the body. The cold gas 92 and the warm gas 95 are generated by a vortex tube 94, e.g., a Hilsch vortex tube. The vortex tube may be driven by a source of compressed gas 93 such as a gas bottle or tank, or a gas compressor, or other source of compressed gas. The cold gas may be supplied to the cooled body portion by a cap or by other systems. The warm gas may be supplied to the warmed body portion via a garment, for example, a vest, leg warmers, socks, booties, slippers, gloves or mittens, sleeves, cuffs, and/or a mask. It is appreciated that the embodiments of FIG. 9 are applicable to temperature control for both heating and cooling in accordance with the embodiments of FIG. 1.

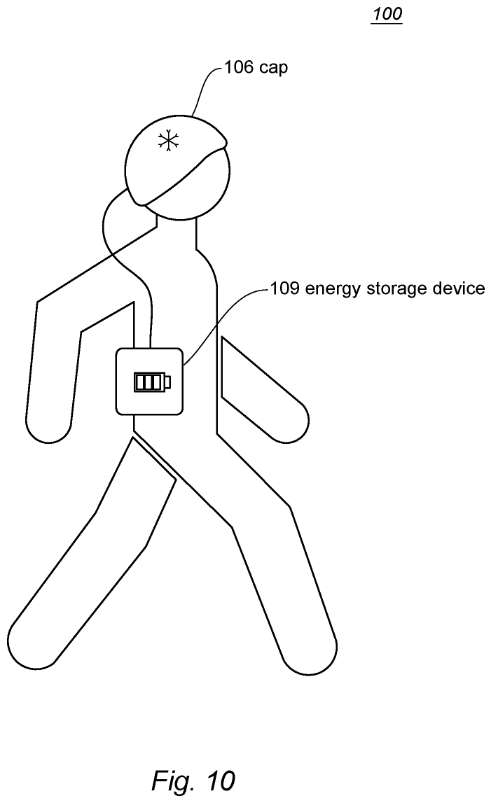

FIG. 10 illustrates a block diagram of an exemplary heating/cooling/compression system 100, in accordance with embodiments of the present invention. System 100 comprises a heating/cooling/compression apparatus 106, e.g., a cooling cap, and an energy storage device 109. The heating/cooling/compression apparatus 106 is configured to conform to a particular body portion, e.g., a human head. The energy necessary to drive a refrigerator, thermoelectric generator, compressor, or pump may be supplied by a battery or other energy storage device 109 that may be transportable with the patient, thus allowing the patient to move over large distances. In some embodiments, the energy storage device 109 may be housed in a backpack, chest pack, waist pack, pocket, and/or other wearable container. In some embodiments, the energy storage device 109 may be attached to a patient, for example, via straps and/or sleeves. In some embodiments, energy may be supplied by a combination of storage device and external power such that the battery will last for a suitable period of time to allow the patient to move. Such a suitable period may be 5, 15, 30, or 60 minutes, or 2, 3, 4, 5, 6, 8, 10, or 12 hours. In some embodiments, a duration of energy supply may correspond with a suitable treatment duration. In some embodiments, a duration of energy supply may correspond with a duration required for chemotherapy drugs to be exhausted from the body, or to decrease to a level not associated with deleterious side effects. In some embodiments, the external power may be supplied by AC mains power, by inductively coupled power, by an automobile power outlet (AC or DC), or by other external power supply.

In some embodiments, energy storage device 199 may power a warming device, e.g., warming apparatus 82 of FIG. 8, in addition to or instead of cooling apparatus 106. In some embodiments, a thermal fluid may be circulated between cooling apparatus 106 and a warming apparatus 82. It is appreciated that the embodiments of FIG. 10 are applicable to temperature control for both heating and/or cooling in accordance with the embodiments of FIG. 1.

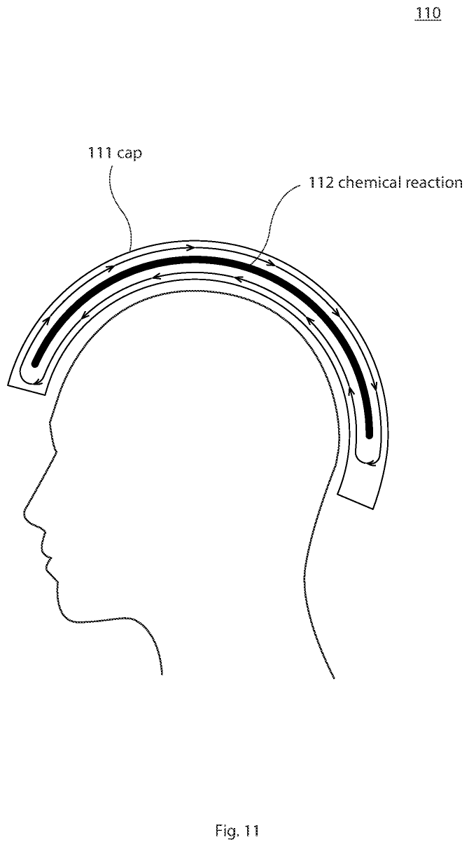

FIG. 11 illustrates a block diagram of an exemplary heating/cooling/compression system 110, in accordance with embodiments of the present invention. System 110 comprises a heating/cooling/compression apparatus 111, e.g., a cooling cap. The heating/cooling/compression apparatus 111 is configured to conform to a particular body portion, e.g., a human head. The inner surface of heating/cooling/compression apparatus 111 may be cooled by an endothermic chemical reaction 112. The endothermic chemical reaction may take place adjacent to the inner surface or may take place removed from the inner surface such that a heat conduction media conducts heat from the inner surface to the endothermic chemical reaction. In some embodiments, an exothermic chemical reaction 112 may be utilized to warm a portion of a body in a complementary fashion. It is appreciated that the embodiments of FIG. 11 are applicable to temperature control for both heating and cooling in accordance with the embodiments of FIG. 1. For example, a flow rate of chemical reactant(s) may be controlled to control an amount of heat produced and/or absorbed by chemical reaction 112.

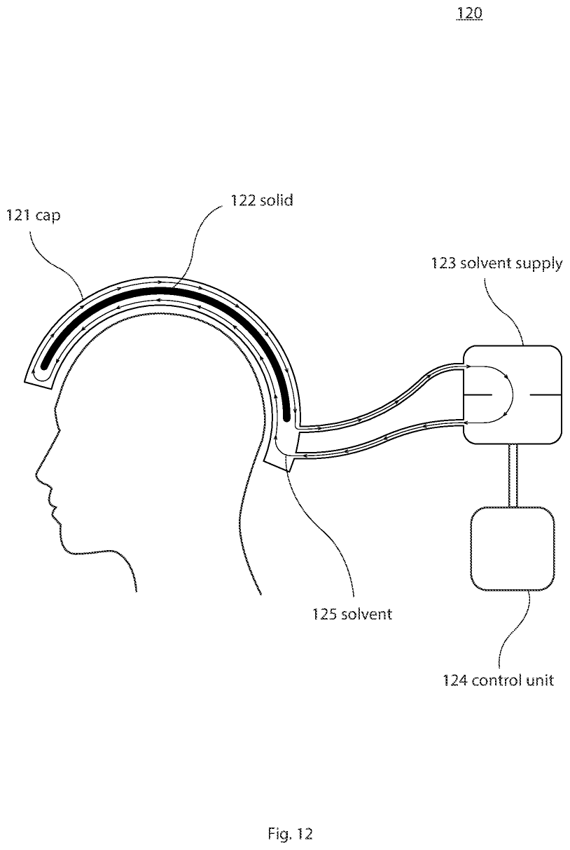

FIG. 12 illustrates a block diagram of an exemplary heating/cooling/compression system 120, in accordance with embodiments of the present invention. System 120 comprises a heating/cooling/compression apparatus 121, e.g., a cooling cap. The heating/cooling/compression apparatus 121 is configured to conform to a particular body portion, e.g., a human head. An endothermic chemical reaction comprises the dissolution of a solid 122, for example, ammonium chloride, by a solvent 125, such as water. In some embodiments, the solid 122 may be contained in the heating/cooling/compression apparatus 121, and the solvent may be supplied to the heating/cooling/compression apparatus 121 by a pump, a pressurized tank, or other systems of supply 123.

In some embodiments, the rate of supply of the solvent 125 may be controlled by a control unit 124 so as to achieve the desired amount of heating/cooling/compression. Such a control unit may be part of a closed-loop controller, e.g., closed loop controller 2 as previously described with respect to FIG. 1. In some embodiments, the heating/cooling/compression apparatus 121, containing the solid 122, may be a single use device such that when the solid 122 may be partially or fully dissolved, the heating/cooling/compression apparatus 121 may be discarded or reprocessed. In some embodiments, the solvent supply 123 and control unit 124 are portable allowing the patient to move from place to place.

In some embodiments, an amount of solid reactant 122 may be set, e.g., sized and/or in an amount provided, to last for a suitable period of time to allow the patient to move. Such a suitable period may be 5, 15, 30, or 60 minutes, or 2, 3, 4, 5, 6, 8, 10, or 12 hours. In some embodiments, an amount of solid reactant 122 may correspond with a suitable treatment duration. In some embodiments, an amount of solid reactant 122 may correspond with a duration required for chemotherapy drugs to be exhausted from the body, or to decrease to a level not associated with deleterious side effects. In some embodiments, solvent supply 123 may be supplied by a coupling to a utility-scale water supply.

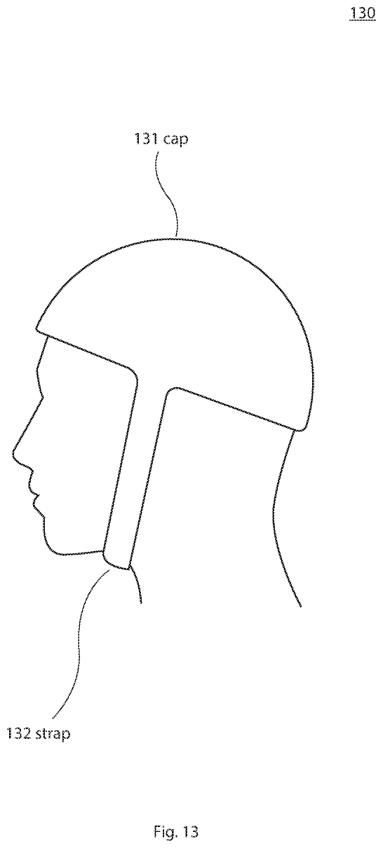

FIG. 13 illustrates a block diagram of an exemplary heating/cooling/compression system 130, in accordance with embodiments of the present invention. System 130 comprises a heating/cooling/compression apparatus 131, e.g., a cooling cap. The heating/cooling/compression apparatus 131 is configured to conform to a particular body portion, e.g., a human head. Heating/cooling/compression apparatus 131 may be cooled by any suitable mechanism. In accordance with embodiments of the present invention, the cap 131 may be retained against the head with strap(s) 132, for example, straps that buckle under the chin, straps that are joined by hook and loop material, or by elastic force such as rubber or other elastomeric material which conforms to the head and by friction retains the cap against the head. In some embodiments, the cap applies pressure to the scalp causing compression of the tissues of and beneath the scalp or skin.

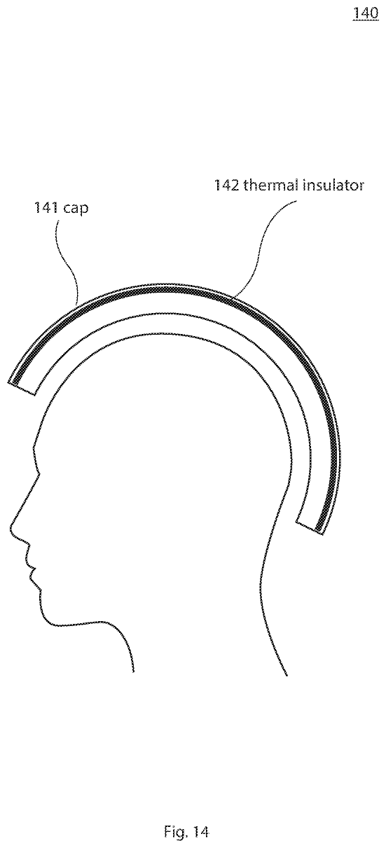

FIG. 14 illustrates a block diagram of an exemplary heating/cooling/compression system 140, in accordance with embodiments of the present invention. System 140 comprises a heating/cooling/compression apparatus 141, e.g., a cooling cap. The heating/cooling/compression apparatus 141 is configured to conform to a particular body portion, e.g., a human head. Heating/cooling/compression apparatus 141 may be cooled by any suitable mechanism. In accordance with embodiments of the present invention, the heating/cooling/compression apparatus 141 comprises a thermal insulator 142 that prevents heat from the environment from interfering with the cooling provided to the scalp. The thermal insulator 142 may comprise a layer that may be disposed outside of the inner surface, opposite to the side proximate to the hair or scalp. In some embodiments, other forms of cooling apparatuses 141, including, for example, a vest, leg warmers, socks, booties, slippers, gloves or mittens, sleeves, cuffs, and/or a mask, may include a thermal insulator comparable to thermal insulator 142 in form and function.

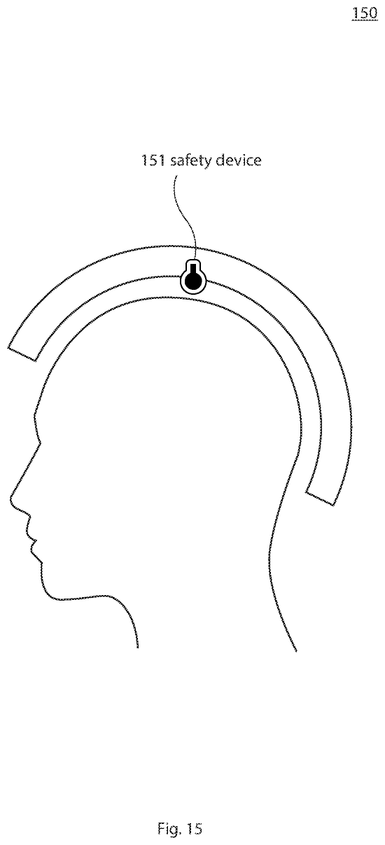

FIG. 15 illustrates a block diagram of an exemplary thermal safety system 150, in accordance with embodiments of the present invention. Thermal safety system 150 comprises a thermal and/or compression safety device 151. Thermal safety device 151 provides safe operation such that a temperature of the skin does not exceed the range of temperature that is safe for the skin of a particular body portion, e.g., the head. Such a safe range of temperature, for example, may lie between −37 degrees Celsius (−35 degrees Fahrenheit) and 48 degrees Celsius (120 degrees Fahrenheit). In some embodiments, thermal safety system 150 may stop a heating, cooling, and/or compression process. In some embodiments, thermal safety system 150 may adjust a heating, cooling, and/or compression process. In some embodiments, thermal safety device 151 may operate within, or be a part of, a closed loop controller, e.g., closed loop controller 2 as described with respect to FIG. 2. In some embodiments, thermal safety device 151 may operate in an open loop manner, e.g., as a fuse, to prevent heating, cooling, and/or compression beyond safe limits. In some embodiments, thermal safety device 151 may stop a chemical reaction, e.g., by closing a valve and/or injecting a reactant to stop such reaction.

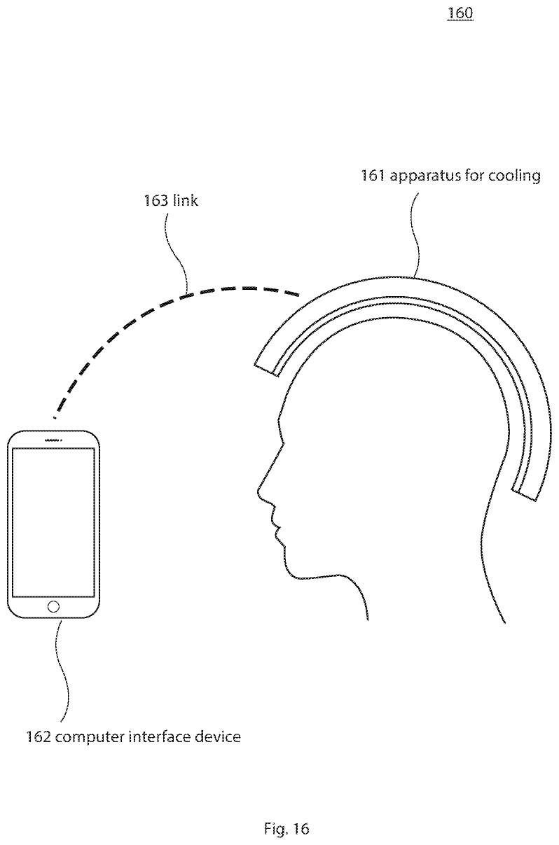

FIG. 16 illustrates a block diagram of an exemplary thermal control system 160, in accordance with embodiments of the present invention. Thermal control system 160 comprises a cooling (or heating) apparatus 161, and a computer interface device 162. Computer interface device 162 may be a held-held computer, e.g., a smart phone, in some embodiments. Computer interface device 162 may be linked 163 to the cooling apparatus 161. The link 163 may be one way only in some embodiments, e.g., the computer interface device 162 sends commands to the cooling apparatus 161. In some embodiments, the link 163 may be two way. For example, the cooling apparatus 161 sends information, e.g., measured skin temperature and/or coolant temperature, to the computer interface device 162, in addition to the computer interface device 162 sending commands to the cooling apparatus 161.

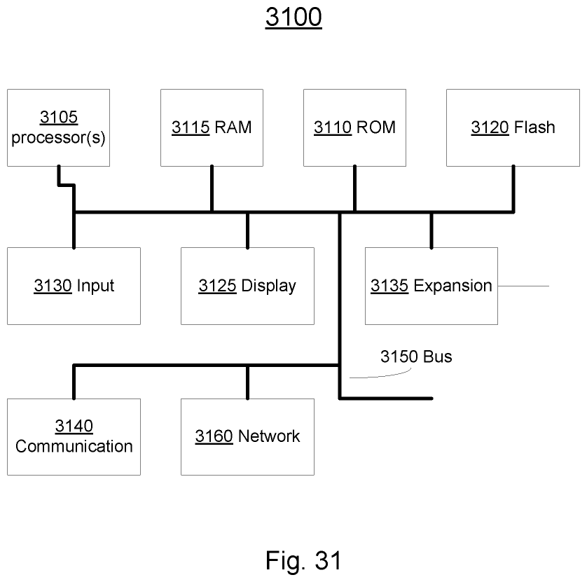

The computer interface device 162 executes software which provides a range of controls for the cooling apparatus 161. Such a computer interface device may be a personal computer, a tablet, a smart phone, a smart TV, or other such interface device. Embodiments of the computer interface device 162 are further described below with respect to FIG. 31, below. The specific cooling (or heating) protocol may be automatically selected based on the patient entering the specific drug being infused, with longer cooling times for drugs with longer blood clearance times. In some embodiments, computer interface device 162 (or a coupled computer system, not shown) may record actual temperatures, e.g., at intervals, during a treatment session. In some embodiments, computer interface device 162 may display real time skin temperature, time elapsed, time remaining, and/or other measured parameters, during a treatment session. In some embodiments, the computer interface device 162 may be part of a closed loop temperature control system, for example, as described with respect to FIG. 1. For example, the computer interface device 162 may serve as a control unit 4 or augment the function of a control unit 4.

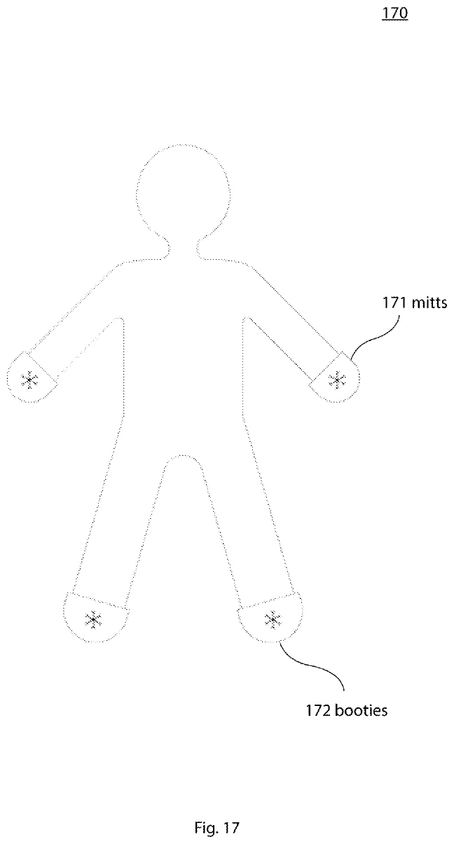

FIG. 17 illustrates a block diagram of an exemplary thermal management system 170, in accordance with embodiments of the present invention. Thermal management system 170 may comprise heating and/or cooling elements for hands and/or fingers, e.g., mitts 171, and/or heating and/or cooling elements for feed and/or toes, e.g., booties 172. The heating/cooling/compression apparatuses 171,172 are configured to conform to a particular body portion, e.g., a human hand and/or feet. The thermal management provided, for example, to the hands, may be configured to allow a patient to continue to use their hands in a normal fashion. In some embodiments, the cooling provided to the hands may be provided preferentially to the tips of the fingers, thus allowing the preservation of nerves going to or from the tips of the fingers. In some embodiments, the mitts 171 and/or booties 172 may provide compression of the tissues of other regions of the body. In some embodiments, thermal management and compression are provided.

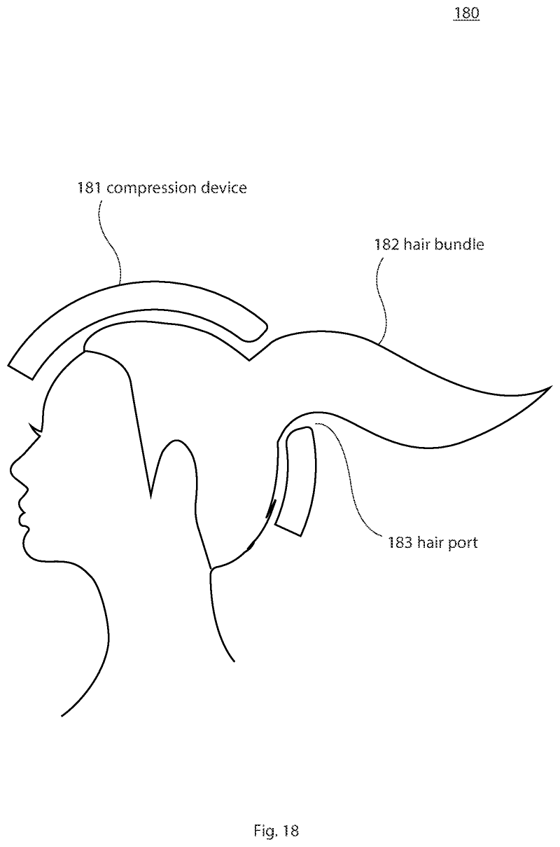

FIG. 18 illustrates a block diagram of an exemplary hair management system 180, in accordance with embodiments of the present invention. Hair management system 180 comprises a compression device 181 with a hole or hair port 183. 18. To monitor the preservation of hair, an embodiment comprises gathering the hair into a bundle 182, e.g., a ponytail, and measuring an aspect of the amount of hair in the bundle. In some embodiments, the bundle may be compressed 181 to a predetermined compression and the area occupied by the hair as it passes through the hair port 183 of compression device 181 may be measured. In some embodiments, the linear dimensions of the area are measured. Such linear dimensions may be a diameter of a circular or nearly circular area, a major axis, and/or a minor axis of an elliptical or nearly elliptical area, or the width and height of a rectilinear or nearly rectilinear area, or any such linear characteristic of the area occupied by the hair. In some embodiments, the amount of hair may be estimated from measurements of the scattering of electromagnetic radiation by the bundle. Such radiation may be visible light, infrared radiation, terahertz radiation, millimeter-wave radiation, microwave radiation, or any other suitable electromagnetic radiation. In some cases, the length of hair may not be sufficient to gather all of the hair into a single bundle. In some embodiments, separate bundles may be created and each of those separate bundles can be measured by systems and methods similar to the above. In some embodiments, the preservation of hair may be estimated by measuring the hair gathered from a portion of the scalp and measuring an aspect of the bundle of the portion of hair as described above. It is appreciated that embodiments in accordance with FIG. 18 are applicable to other areas of hair in addition to the scalp, including, for example, facial hair, e.g., beards, and/or body hair.

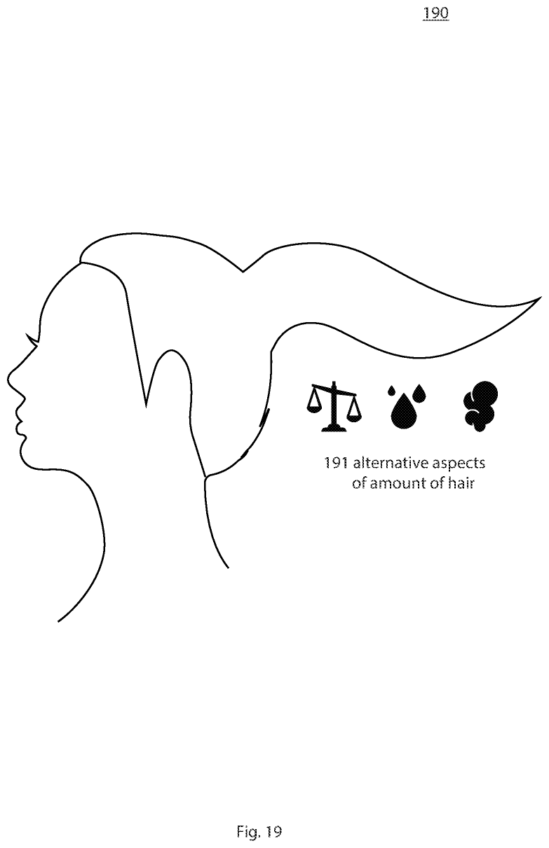

FIG. 19 illustrates a block diagram of an exemplary hair management system 190, in accordance with embodiments of the present invention. In accordance with embodiments of the present invention, an amount of hair may be estimated by measuring alternative aspects 191 of the amount of hair. In some embodiments, the aspect may be the weight of the hair. In this case, the weight must be measured such that the weight of the remainder of the patient's body, such as the weight of the head, does not confound the measurement of the weight of the hair. In some embodiments, the aspect may be the weight or volume of water taken up by the hair when the hair may be wetted starting from a state of dryness. In some embodiments, the aspect of the amount of hair may be the ease with which a puff or stream of air or other gas or other fluid displaces or otherwise disturbs the hair.

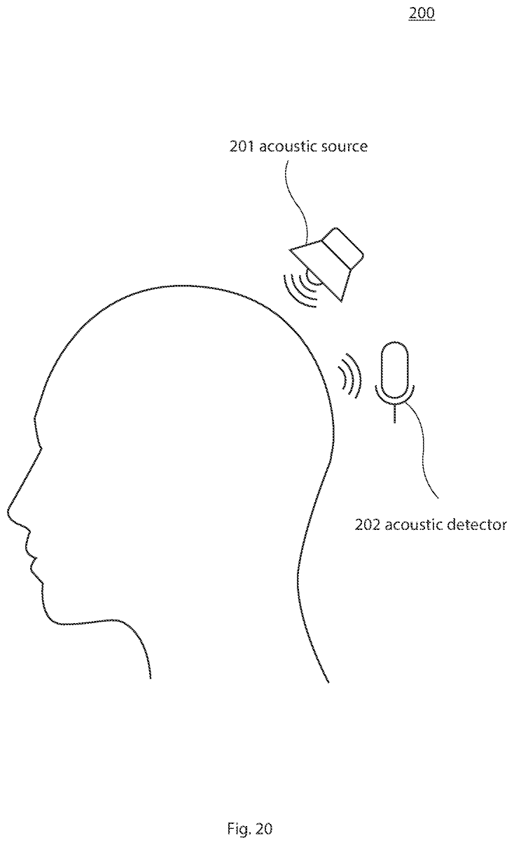

FIG. 20 illustrates a block diagram of an exemplary hair management system 200, in accordance with embodiments of the present invention. In accordance with embodiments of the present invention, the aspect may be the damping or other modification of acoustic energy propagated through the hair. In some embodiments, acoustic energy may be aimed at the head from an acoustic source 201 and the amount of acoustic energy reflected by the head may be measured by an acoustic detector 202. The hair will damp the acoustic waves as they propagate first toward the head and then away from the head. The degree of damping will be related to the amount of hair and the structural properties of the hair. A change in the preservation of hair may be inferred from such a change in the degree of damping of the reflected acoustic waves. In some embodiments, the acoustic detector and the acoustic source comprise the same equipment, and the two functions are facilitated by first generating the acoustic energy, for example as a pulse, and then detecting the return pulse. In some embodiments, acoustic energy may be aimed to pass through the hair but not reflect off the head. This may be accomplished by, for example, allowing the hair to hang freely from the head. The preservation of hair may be inferred from amount of acoustic energy transmitted through the hair, measured by an acoustic detector disposed opposite the acoustic source relative to the hair.

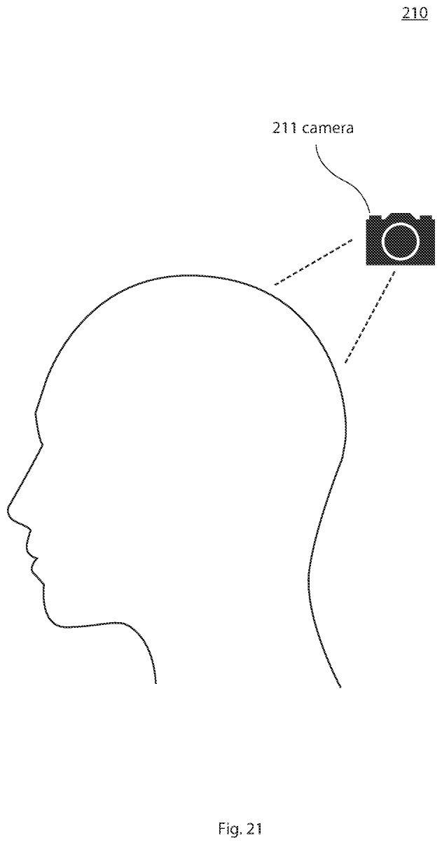

FIG. 21 illustrates a block diagram of an exemplary hair management system 210, in accordance with embodiments of the present invention. In accordance with embodiments of the present invention, the visual density of the hair may be measured. A camera 211 may be used to capture one or more images of the hair. Analysis software can assess how much hair may be present in the captured images by, for example, counting hair strands. This may be done either close to the hair or from a substantial distance away.

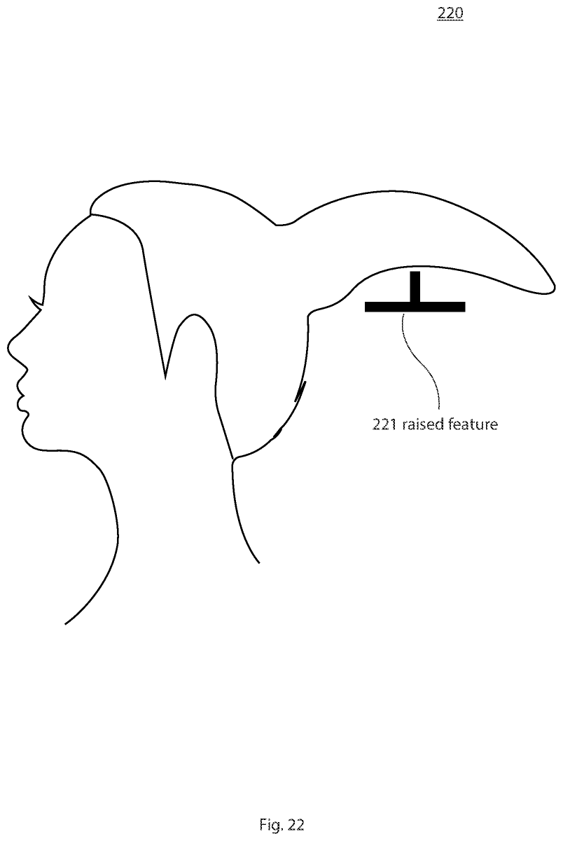

FIG. 22 illustrates a block diagram of an exemplary hair management system 220, in accordance with embodiments of the present invention. In accordance with embodiments of the present invention, the preservation of hair may be monitored by measuring mechanical aspects of the hair. In one such embodiment, the bending of the hair may be measured by allowing the hair to lie across a substantially horizontal surface comprising a raised feature 221. The raised feature 221 may be a thin plate projecting substantially vertically from the surface, similar to a fence rising from a field. The hair may be allowed to drape across the plate and the shape that the hair takes may be analyzed to yield the tendency of the hair to remain straight under bending. The strength of the hair and therefore the preservation of the hair may be estimated from such an analysis of bending.

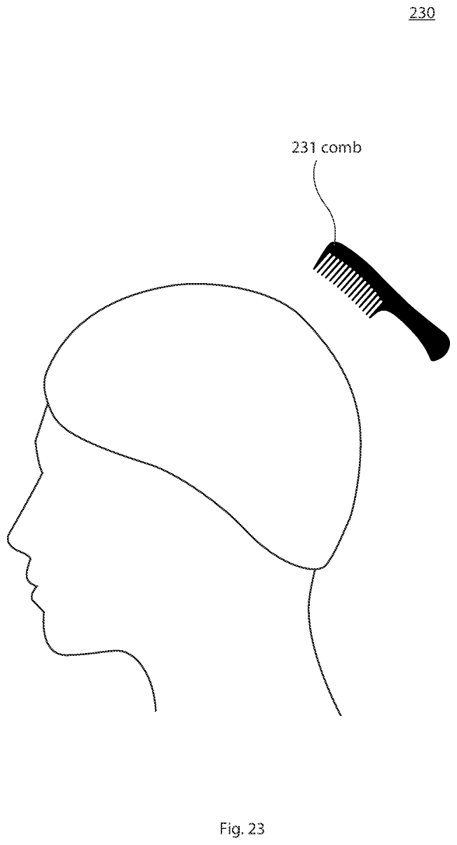

FIG. 23 illustrates a block diagram of an exemplary hair management system 230, in accordance with embodiments of the present invention. In accordance with embodiments of the present invention, the preservation of hair may be monitored by measuring the interaction of hair with the tines of a comb 231 or comb-like device. The interaction may be the resistance, expressed for example as a force, of the hair as the comb may be pulled through the hair. In some embodiments, the tines may be equipped with optical or acoustic sensors which measure the density of hair between them.

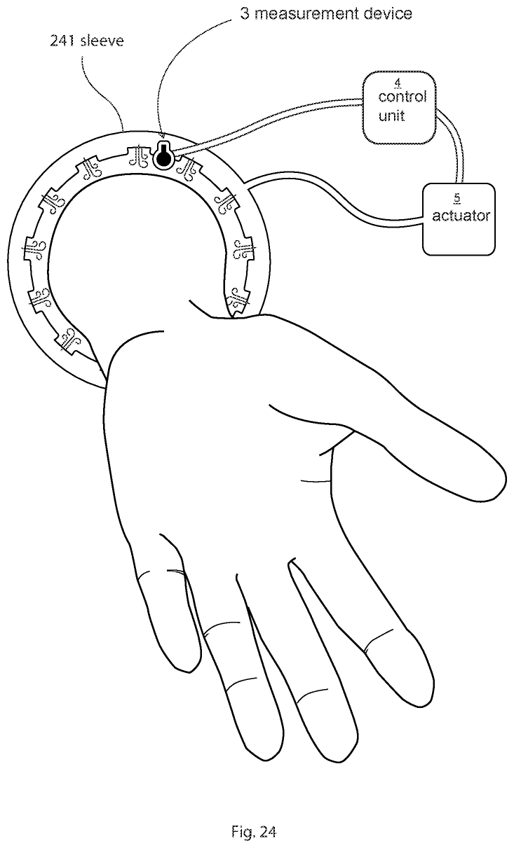

FIG. 24 illustrates a block diagram of an exemplary closed loop controller 2 for use with a heating/cooling/compression apparatus, e.g., a cooling sleeve 241, in accordance with embodiments of the present invention. Controller 2 comprises a measurement device 3 embedded in cooling sleeves 241. The measurement device 3 may comprise a thermistor, a fiber-optic thermometer, a non-contact thermometer, e.g., an infrared thermometer, or other temperature measurement device(s). The measurement device may measure the temperature of the skin, the temperature of the medium, such as air or a fluid or gel proximate to the skin, the temperature of hair, the temperature of a covering or garment proximate to the skin, or any combination of these.

In accordance with embodiments of the present invention, measurement device 3 may sense properties of tissue being cooled, for example, rather than or in addition to directly sensing temperature. Such a sensor may sense blood flow, pulse and/or pulse rate, a presence or absence of substances, cellular metabolism, e.g., via NADH (nicotinamide adenine dinucleotide and hydrogen) fluorescence sensors, pressure and/or compression, or any other suitable sensed property. Sensed substances may include chemotherapy agents and/or their metabolites, or other substances indicative of tissue status or tissue metabolism. The output from any such sensor may be incorporated into control such that changes in a sensed property cause corresponding changes in the control of cooling, heating, and/or compression. Such incorporation of the output of any such sensor into control may allow control to alter cooling, heating, and/or compression, to avoid unsafe conditions for such tissue being cooled. In some embodiments sensors may sense properties of the head or the tissues of limbs such as arms, legs, hands, feet, fingers, face, eyebrows, toes, lips, and/or mouth, or any combination thereof.

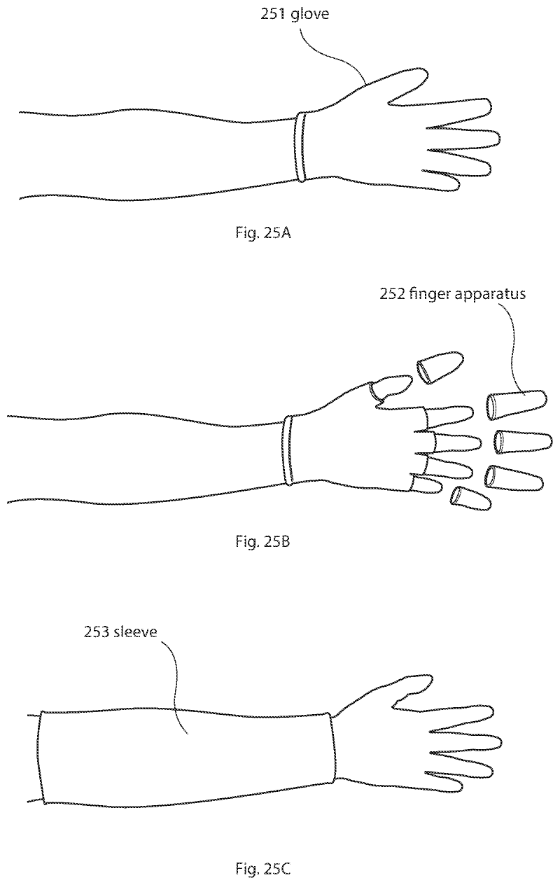

FIGS. 25A, 25B, and 25C illustrate exemplary systems of heating, cooling, and/or compressing hands, fingers and arms, respectively, in accordance with embodiments of the present invention. In accordance with embodiments of the present invention, heating, cooling, and/or compression may be provided to the hands and/or fingers and/or arms collectively, in combination and/or in isolation. As illustrated in FIG. 25A, heating, cooling, and/or compression is/are applied by a glove 251 with each finger covered independently, with closed fingers or any other form that has substantially one opening. In some embodiments, the hand and/or wrist may not be heated, cooled and/or compressed. As illustrated in FIG. 25B, heating, cooling and/or compression may be applied to one or more fingers individually, e.g., via individual finger apparatus 252. For example, not all fingers may be cooled. In some embodiments, different fingers may be controlled to different temperatures or other criteria, e.g., blood flow or presence of a chemotherapy drug. As illustrated in FIG. 25c, heating, cooling, and/or compression may be applied to an arm or arm portion by a sleeve 253 or a cuff or a fingerless glove or any other form that has substantially more than one opening.

In accordance with embodiments of the present invention, the heating, cooling and/or compression apparatus may provide the ability for the patient to use the fingers or hands or arms for any normal use thereof. Such uses may include writing, typing, using a telephone including, for example, a smart phone, operating devices, operating controls of an automobile, handling a book, handling food, assisting with bodily functions, or any other normal use. In some embodiments, the ability to use the fingers or hands may comprise a glove or gloves with removable fingertips, e.g., individual finger apparatus 252.

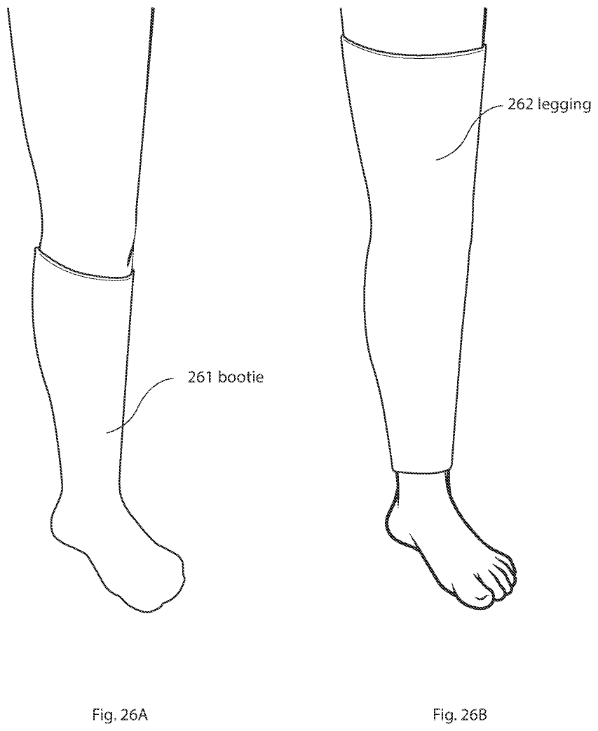

FIGS. 26A and 26B illustrate exemplary systems of heating, cooling, and/or compressing legs and/or feet, in accordance with embodiments of the present invention. In accordance with embodiments of the present invention, heating, cooling, and/or compression may be provided to the feet or legs. In some embodiments heating, cooling and/or compression is/are applied by a bootie 261 or shoe or sock with closed toes or any other form that has substantially one opening. In some embodiments, heating, cooling, and/or compression may be applied to one or more toes only, for example, without applying therapy to other portions of a foot or leg. In some embodiments, heating, cooling, and/or compression may be applied to individual toes. In some embodiments, heating, cooling and/or compression may be applied to one or more toes individually, e.g., via individual toe fittings, similarly to finger apparatus 252 of FIG. 25B. For example, not all toes may be cooled. In some embodiments, different toes may be controlled to different temperatures or other criteria, e.g., blood flow or presence of a chemotherapy drug. In some embodiments heating, cooling and/or compression is/are applied by a legging 262 or sleeve or a cuff or a toeless boot or toeless shoe or toeless sock or any other form that has substantially more than one opening.

In accordance with embodiments of the present invention, the heating, cooling and/or compression apparatus may provide the ability for the patient to use the feet or legs for any normal use thereof. Such uses may include standing, walking, running, peddling a bicycle, operating controls of an automobile, or any other normal use. In some embodiments, an ability to use the feet or legs may comprise a boot or boots. Such a boot or boots may allow simultaneously for using the feet or legs and for heating, cooling, and/or compression.

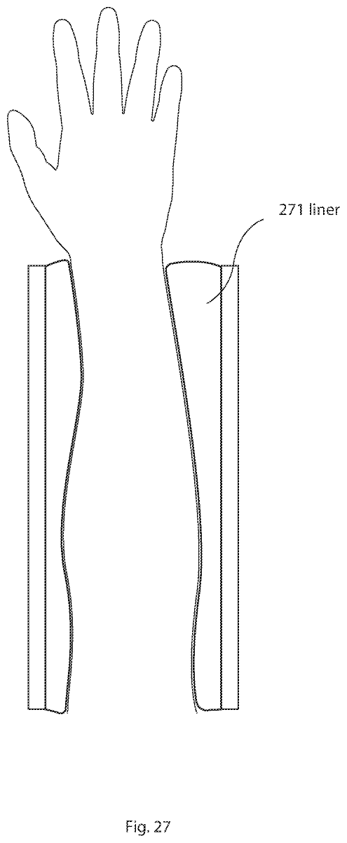

FIG. 27 illustrates an exemplary heating/cooling/compression apparatus liner 271, in accordance with embodiments of the present invention. Liner 271 may be a part of any heating/cooling/compression apparatus, including, for example, a cooling cap, e.g., cooling cap 6 (FIG. 1), bootie 261 (FIG. 26A), legging 262 (FIG. 26B), glove 251 (FIG. 25A), individual finger apparatus 252 (FIG. 25B), sleeve 253 (FIG. 25C), sleeve 241 (FIG. 24), and/or mitts 171 (FIG. 17). In accordance with embodiments of the present invention, liner 271 may be positioned substantially immediately adjacent to the skin of a body part. Liner 271 may be characterized as having a high thermal conductivity to allow rapid transfer of heat from the skin. In some embodiments, liner 271 may be characterized as having a low thermal conductivity to prevent excessive transfer of heat from the skin and thus to avoid an unsafe situation for the skin. The liner 271 may cause compression and protect the skin from damage due to cold and/or heat. In some embodiments, liner 271 may be replaceable, removable, and/or disposable. In some embodiments, liner 271 may help to prevent contamination from previous patients of a heating/cooling/compression apparatus.

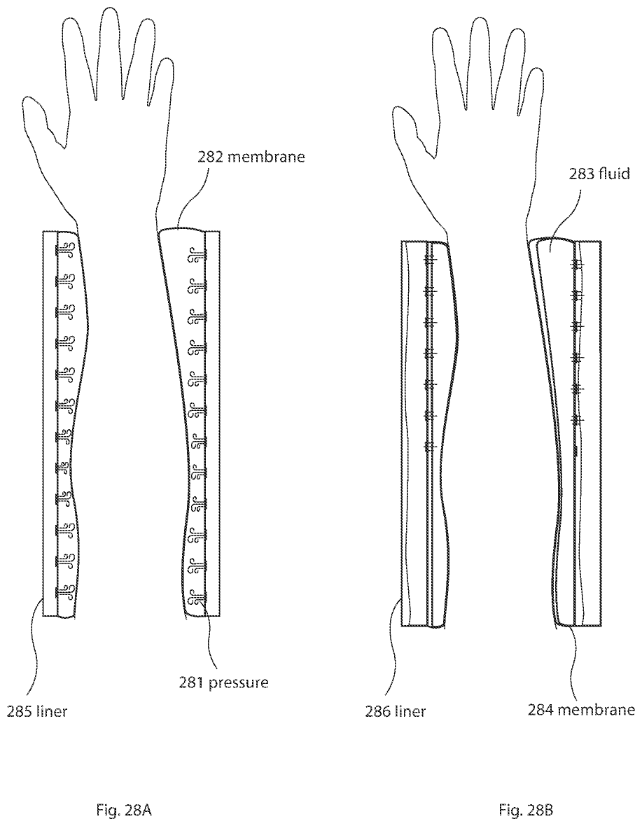

FIGS. 28A and 28B illustrate exemplary liners 285 and 286, respectively, in accordance with embodiments of the present invention. In accordance with embodiment of the present invention, liners 285, 286 may comprise a flexible polymer or a flexible fabric or any other flexible membrane such that it tends to conform to the shape of the body part. Such conformation may be enhanced by the application of pressure 281 to the surface of the liner that is not directly adjacent to the skin. Liner 286 may be substantially impervious to a fluid 283 such as air, water, a gas, a liquid, or any other such fluid. Such an impervious liner may allow pressure to be applied by any such fluid while not allowing that fluid to pass through the liner. Liners 285, 286 may comprise membranes 282, 283 respectively to apply therapeutic compressive force to a body portion.

In some embodiments, liners 285, 286 may provide a barrier to contamination that may impinge on the adjacent skin from the environment. Such contamination may include chemicals or infectious agents or any other contamination. Liners 285, 286 may be washable, single-use, single-patient-use, or disposable so as to avoid transmitting contamination from one patient to another patient.

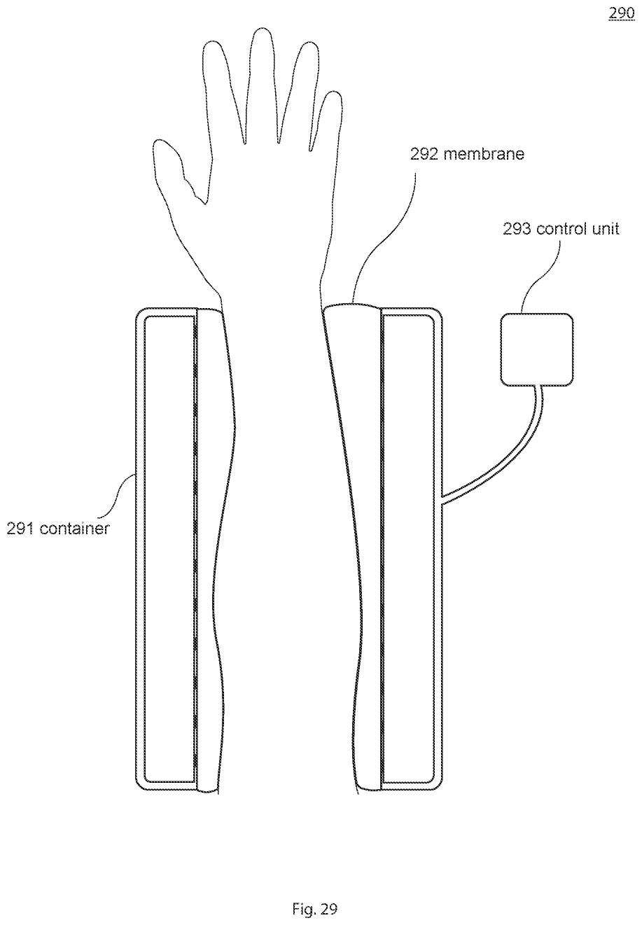

FIG. 29 illustrates an exemplary heating/cooling/compression apparatus 290, in accordance with embodiments of the present invention. Apparatus 290 comprise an outer container 291. Outer container 291 may be rigid, for example, resistant to changes in shape, in some embodiments. Outer container 291, together with a liner may circumscribe a substantially closed volume. Such a volume may be filled with a fluid such as air, or other gas, or water or other liquid. Such a fluid may provide cooling by being introduced at a temperature below the temperature of the adjacent skin. Such a fluid may provide heating by being introduced at a temperature above the temperature of the adjacent skin. Such a fluid may also provide compression by being introduced at a pressure above the local atmospheric pressure. Any combination of temperature and pressure of fluid may be provided. In some embodiments, apparatus 290 may comprise a membrane 292, configured to apply therapeutic compressive force to a body portion.

In some embodiments, the temperature and/or pressure at which the fluid may be provided may be controlled by a controller 293. In some embodiments, control of the temperature and pressure of the introduced fluid are controlled to achieve a therapeutic combination. Such a therapeutic combination may provide lowered level of pain or discomfort as perceived by the patient. Alternatively, such a therapeutic combination may provide higher levels of efficacy as to the reduction of harm to the hands or feet due to the treatment being received by the patient. Such a therapeutic combination may provide lowered pain and/or decreased discomfort, as well as higher levels of efficacy.

In accordance with embodiment of the present invention, heating, cooling and/or compression may be provided to any combination of body parts including the scalp, fingers, toes, hands, feet, arms, legs, mouth, lips, tongue, and/or eyebrows.

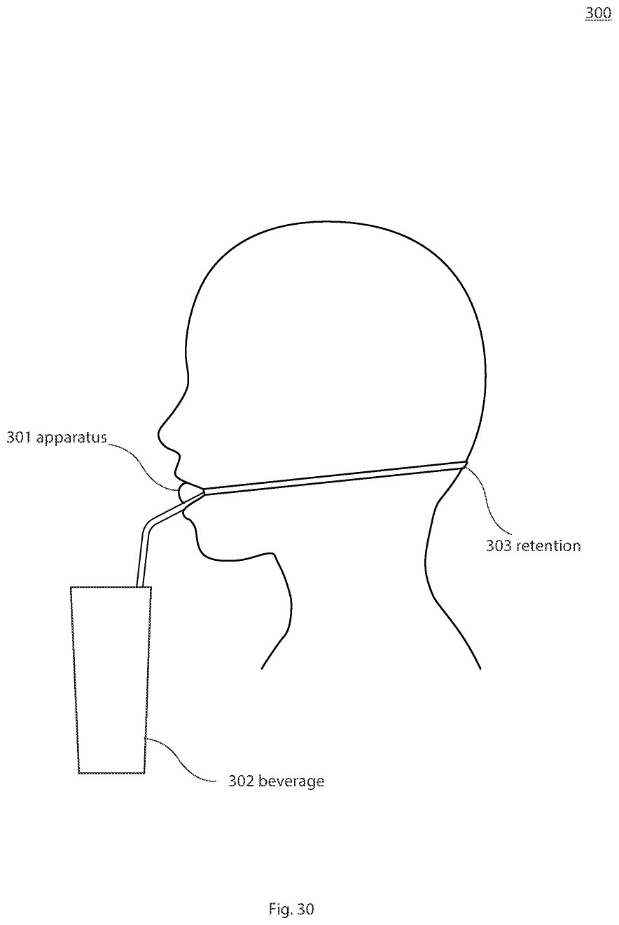

FIG. 30 illustrates an exemplary heating/cooling/compression apparatus 300, in accordance with embodiments of the present invention. Apparatus 300 provides heating, cooling, and/or compression to anatomical components of the mouth in any combination, including, for example, the lips, the gingiva (gums), the buccal mucosa (lip and cheek lining), the floor of the mouth, the tongue, the hard palate, and/or the soft palate, in order to prevent or mitigate chemotherapy side effects, including chemotherapy-induced mucositis. Apparatus 300 may comprise any suitable combination of heating, cooling and/or compression mechanisms, including those mechanisms described elsewhere herein. Apparatus 300 may comprise any suitable combination of sensors or sensing mechanisms including those sensors and sensing mechanisms described elsewhere herein. Apparatus 300 may comprise any suitable combination of control systems for heating, cooling, and/or compression, including those control systems and methods described elsewhere herein.

Apparatus 300 may comprise control mechanism(s) in such a way that the patient or patient's caregiver can manually make adjustments to heating, cooling, and/or compression. Apparatus 300 may comprise control mechanisms such that the device automatically sets therapy time based on an infused drug. For example, a patient may enter and/or select the name of the infused drug via an interface device, e.g., computer interface device 162 as described with respect to FIG. 16. Responsive to the drug information, apparatus 300 may automatically set a therapy time. In some embodiments, apparatus 300 may assign longer therapy times to drugs with longer blood clearance times. Apparatus 300 may comprise control mechanisms such that the device automatically adjusts heating, cooling, and/or compression in response to sensors or sensing incorporated in the device. Apparatus 300 may comprise any suitable structures 303, e.g., a head strap, for retaining or holding the device in place in a patient's mouth. Apparatus 300 may comprise features to allow a patient to consume a liquid beverage 302 with apparatus 300 in place within the patient's mouth.

FIG. 31 illustrates a block diagram of an exemplary electronic system 3100, which may be used as a platform to implement and/or as a control system for embodiments of the present invention. Electronic system 3100 may be a “server” computer system, in some embodiments. Electronic system 3100 includes an address/data bus 3150 for communicating information, a central processor complex 3105 functionally coupled with the bus for processing information and instructions. Bus 3150 may comprise, for example, a Peripheral Component Interconnect Express (PCIe) computer expansion bus, industry standard architecture (ISA), extended ISA (EISA), MicroChannel, Multibus, IEEE 796, IEEE 1196, IEEE 1496, PCI, Computer Automated Measurement and Control (CAMAC), MBus, Runway bus, Compute Express Link (CXL), and the like.

Central processor complex 3105 may comprise a single processor or multiple processors, e.g., a multi-core processor, or multiple separate processors, in some embodiments. Central processor complex 3105 may comprise various types of well-known processors in any combination, including, for example, digital signal processors (DSP), graphics processors (GPU), complex instruction set (CISC) processors, reduced instruction set (RISC) processors, and/or very long word instruction set (VLIW) processors. Electronic system 3100 may also includes a volatile memory 3115 (e.g., random access memory RAM) coupled with the bus 3150 for storing information and instructions for the central processor complex 3105, and a non-volatile memory 3110 (e.g., read only memory ROM) coupled with the bus 3150 for storing static information and instructions for the processor complex 3105. Electronic system 3100 also optionally includes a changeable, non-volatile memory 3120 (e.g., NOR flash) for storing information and instructions for the central processor complex 3105 which can be updated after the manufacture of system 3100. In some embodiments, only one of ROM 3110 or Flash 3120 may be present.

Also included in electronic system 3100 of FIG. 31 is an optional input device 3130. Device 3130 can communicate information and command selections to the central processor 3100. Input device 3130 may be any suitable device for communicating information and/or commands to the electronic system 3100. For example, input device 3130 may take the form of a keyboard, buttons, a joystick, a track ball, an audio transducer, e.g., a microphone, a touch sensitive digitizer panel, eyeball scanner, and/or the like.

Electronic system 3100 may comprise a display unit 3125. Display unit 3125 may comprise a liquid crystal display (LCD) device, cathode ray tube (CRT), field emission device (FED, also called flat panel CRT), light emitting diode (LED), plasma display device, electro-luminescent display, electronic paper, electronic ink (e-ink) or other display device suitable for creating graphic images and/or alphanumeric characters recognizable to the user. Display unit 3125 may have an associated lighting device, in some embodiments.

Electronic system 3100 also optionally includes an expansion interface 3135 coupled with the bus 3150. Expansion interface 3135 can implement many well known standard expansion interfaces, including without limitation the Secure Digital Card interface, universal serial bus (USB) interface, Compact Flash, Personal Computer (PC) Card interface, CardBus, Peripheral Component Interconnect (PCI) interface, Peripheral Component Interconnect Express (PCI Express), mini-PCI interface, IEEE 1394, Small Computer System Interface (SCSI), Personal Computer Memory Card International Association (PCMCIA) interface, Industry Standard Architecture (ISA) interface, RS-232 interface, and/or the like. In some embodiments of the present invention, expansion interface 3135 may comprise signals substantially compliant with the signals of bus 3150.

A wide variety of well-known devices may be attached to electronic system 3100 via the bus 3150 and/or expansion interface 3135. Examples of such devices include without limitation rotating magnetic memory devices, flash memory devices, digital cameras, wireless communication modules, digital audio players, and Global Positioning System (GPS) devices.

System 3100 also optionally includes a communication port 3140. Communication port 3140 may be implemented as part of expansion interface 3135. When implemented as a separate interface, communication port 3140 may typically be used to exchange information with other devices via communication-oriented data transfer protocols. Examples of communication ports include without limitation RS-232 ports, universal asynchronous receiver transmitters (UARTs), USB ports, infrared light transceivers, ethernet ports, IEEE 1394, and synchronous ports.

System 3100 optionally includes a network interface 3160, which may implement a wired or wireless network interface. Electronic system 3100 may comprise additional software and/or hardware features (not shown) in some embodiments.

Various modules of system 3100 may access computer readable media, and the term is known or understood to include removable media, for example, Secure Digital (“SD”) cards, CD and/or DVD ROMs, diskettes and the like, as well as non-removable or internal media, for example, hard drives, solid state drive s (SSD), RAM, ROM, flash, and the like.

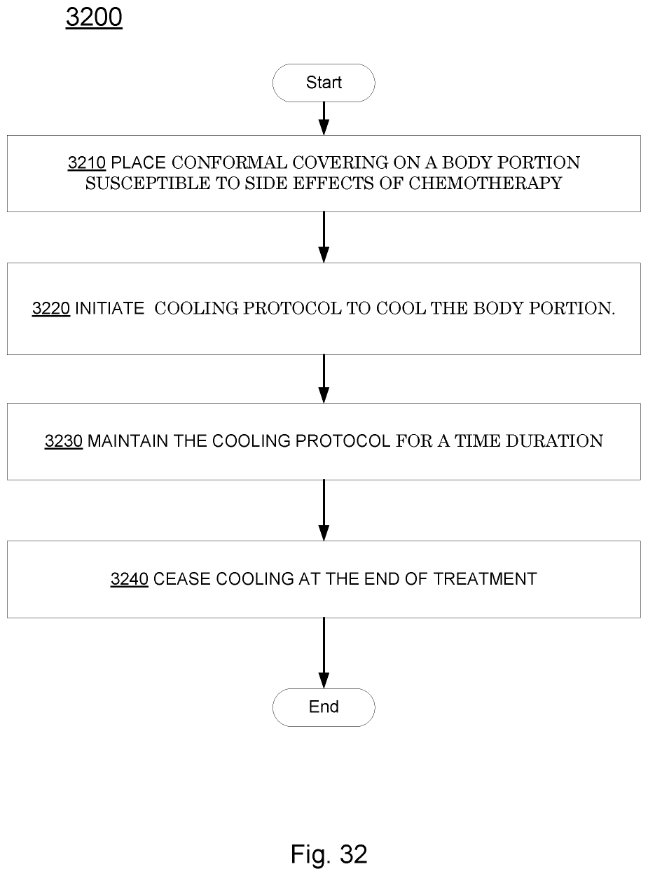

FIG. 32 illustrates an exemplary method 3200 for mitigating side effects of chemotherapy, in accordance with embodiments of the present invention. In 3210, conformal covering, for example, cooling cap 6 (FIG. 1) and/or glove 251 (FIG. 25A), is placed on a body portion susceptible to side effects of chemotherapy, e.g., the head. In 3220, a cooling protocol is initiated, cooling the body portion. In some embodiments, the cooling protocol may include a ramp in temperature from ambient temperature, to avoid thermal shock to the body portion. In some embodiments, the cooling may be tailored to a specific drug treatment. In some embodiments, the cooling may be based on a detected level of a drug within the body portion.

In 3230, the cooling protocol is maintained for a time duration. The cooling protocol may be controlled by control unit 4 (FIG. 1), computer interface device 162 (FIG. 16), and/or electronic system 3100 (FIG. 31), for example. In some embodiments, the temperature of the body portion may be held constant, and/or may be varied in accordance with sensed aspect of the body portion. In some embodiments, cellular metabolism may be sensed. In some embodiments, blood flow may be sensed. In 3240, cooling of the body portion is ceased at the end of treatment. The end of treatment may be determined by elapsed time of treatment and/or any sensed aspect of the body portion, for example, presence, or lack thereof, of a treatment drug. The cooling may be ramped from a therapeutic temperature to ambient temperature, to avoid thermal shock to the body portion.

Embodiments in accordance with the present invention provide systems and methods for mitigating side effects of chemotherapy. In addition, embodiments in accordance with the present invention provide systems and methods for mitigating side effects of chemotherapy that are portable and allow a patient to walk during treatment. Further, embodiments in accordance with the present invention provide systems and methods for mitigating side effects of chemotherapy that provide a patient and/or caregiver with temperature and/or pressure control. Still further, embodiments in accordance with the present invention provide systems and methods for mitigating side effects of chemotherapy that are compatible and complementary with existing systems and methods of administering chemotherapy.

Although the invention has been shown and described with respect to a certain exemplary embodiment or embodiments, equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (e.g., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

Various embodiments of the invention are thus described. While the present invention has been described in particular embodiments, it should be appreciated that the invention should not be construed as limited by such embodiments, but rather construed according to the below claims.

1. A therapeutic cooling system configured to cool a body portion, the cooling system comprising:
a conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301) for covering said body portion configured to extract heat energy from said body portion;
a sensor device (3, 151) within said conformal covering for sensing a parameter of said body portion;
an actuator (5, 22) configured for changing an amount of heat energy extracted from said body portion;
a control unit (4, 44, 124, 162, 3100) configured for regulating said actuator (5, 22) responsive to control input from said sensor device (3, 151).

2. The therapeutic cooling system of claim 1 wherein said sensor device (3, 151) measures temperature proximate to said body portion, measures a body metabolism condition of said body portion, or measures a body metabolism condition of said body portion.

3. (canceled)

4. (canceled)

5. The therapeutic cooling system of claim 1 wherein said control unit (4, 44, 124, 162, 3100) is further configured to adjust an amount of heat extracted from said body portion based on said presence of a therapeutic drug in said body portion, based on manual input, or based on an amount of hair between a skin surface and said conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301).

6. (canceled)

7. (canceled)

8. The therapeutic cooling system of claim 1 wherein said control unit (4, 44, 124, 162, 3100) is further configured to adjust an amount of heat extracted from said body portion to maintain a safe temperature of said body portion.

9. The therapeutic cooling system of claim 1 further comprising a safety device (151) configured to adjust cooling of said body portion in order to maintain a safe temperature of said body portion.

10. The therapeutic cooling system of claim 1 wherein said actuator (5, 22), said control unit (4, 44, 124, 162, 3100) and a source of cooling are secured to a patient to enable said patient to walk during treatment.

11. The therapeutic cooling system of claim 1 wherein said body portion includes a hand, and said conformal covering (241, 251, 252, 253, 261, 262, 271, 282, 284, 292, 301) is configured to allow use of said hand.

12. The therapeutic cooling system of claim 1 wherein said conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301) is further configured to apply compression to said body portion.

13. The therapeutic cooling system of claim 1 further comprising:
a second conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301) for covering a second body portion configured to extract heat energy from said a second body portion, wherein said second conformal covering is configured to operate independently of said first conformal covering.

14. The therapeutic cooling system of claim 1 wherein said conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301) is configured to cool one or more fingers or toes in isolation from other body portions.

15. (canceled)

16. A therapeutic thermal management system configured to cool a first body portion and warm a second body portion, the system comprising:
a first conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301) for covering said first body portion, configured to extract heat energy from said first body portion;
a second conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301) for covering said second body portion, configured to apply heat energy from said second body portion;
first and second sensor devices (3, 151) within said first and said second conformal coverings for sensing a parameter of said first and second body portions;
an actuator (5, 22) configured for changing an amount of heat energy extracted from said first body portion and applied to said second body portion;
a control unit (4, 44, 124, 162, 3100) configured for regulating said actuator (5, 22) responsive to control input from said first and second sensor devices (3, 151).

17. The therapeutic thermal management system of claim 16 wherein heat extracted from said first body portion is applied to said second body portion.

18. The therapeutic thermal management system of claim 16 wherein at least one of said first and second sensor devices (3, 151) is configured to measure cellular metabolism.

19. The therapeutic thermal management system of claim 16 further comprising a replaceable liner (271, 285, 286).

20. The therapeutic thermal management system of claim 18 wherein said a control unit (4, 44, 124, 162, 3100) is further configured to ramp at least one of said first and second conformal coverings from ambient temperature to a therapeutic temperature to prevent shock to a patient's skin.

21. The therapeutic thermal management system of claim 16 wherein said a control unit (4, 44, 124, 162, 3100) is further configured to control at least one of said first and second conformal coverings from zero pressure to a therapeutic pressure to prevent shock to a patient's skin.

22. The therapeutic thermal management system of claim 16 wherein said a control unit (4, 44, 124, 162, 3100) is further configured to accept manual control inputs from a portable computer device (162, 3100) to control a desired temperature of said first and/or said second conformal covering, wherein optionally said portable computer device (162, 3100) is further configured to display elapsed therapeutic time and remaining therapeutic time and/or configured to accept user input of a specific drug therapy and responsive to said input automatically initiate an appropriate cooling protocol for said specific drug therapy.

23. (canceled)

24. (canceled)

25. A therapeutic cooling system configured to cool a body portion, the cooling device comprising:
a conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301) for covering said body portion configured to extract heat energy from said body portion;
a cooling device (23, 32, 45, 53, 62, 72, 94, 121) configured to cool said conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301);
a sensor device (3, 151) within said conformal covering for sensing a parameter of said body portion;
an actuator (5, 22) configured for changing an amount of heat energy extracted from said body portion;
a control unit (4, 44, 124, 162, 3100) configured for regulating said actuator (5, 22) responsive to control input from said sensor device (3, 151).

26. The therapeutic cooling system of claim 25 wherein said cooling device comprises a vortex tube (94), a closed loop heat pump (72), a remote radiator (53), a pre-cooled gas (32), a thermoelectric cooler (45), or a source of gas at ambient temperature and cooling is effected by expansion of said gas (23).

27. (canceled)

28. (canceled)

29. (canceled)

30. (canceled)

31. (canceled)

32. The therapeutic cooling system of claim 25 wherein said cooling device (23, 32, 45, 53, 62, 72, 94, 121) is a part of said conformal covering (6, 26, 36, 43, 51, 63, 71, 81, 82, 91, 96, 106, 111, 121, 131, 141, 161, 171, 172, 241, 251, 252, 253, 261, 262, 271, 282, 284, 292, 301).
